(19) United States
(12) Patent Application Publication
(10) Pub. No.: US 2004/0122287 A1
(43) Pub. Date: Jun. 24, 2004

(54) Human female facsimile

(76) Inventor: Phillip V. Minigh, East Bend, NC (US)

Correspondence Address: Phillip V. Minigh, P O. Box 1503, Winston-Salem, NC, 27116, US

(21) Appl. No.: 10/326,926

(22) Filed: Dec. 23, 2002

(51) Int. Cl. A61F 5/00

(52) U.S. Cl. 600/38

(57) ABSTRACT

A human female facsimile is an anatomically correct appliance that is made with a rigid skeletal system consisting of PVC or similar polymer support structure and articulated joints to assimilate the human range of movement. Covering the skeletal system is a tissue layer made of polyurethane or other similar polymer that approximates the density and feel of the human body. The outer or skin covering is made of silicone or other similar poly elastomer, with the elasticity, texture and feel of human skin. The appliance is equipped with three orifices as the human female to accept an external cylindrical object, such as the human male penis.

### CROSS-REFERENCED TO RELATED APPLICATIONS

| U.S. Patent | | | |
|---|---|---|---|
| Documents | | | |
| 2763070 | September 1956 | McCormick | 434/273 |
| 4683669 | September 1987 | Greer, Jr. | 40/414 |
| 5466235 | November 1995 | Shubin, Sr. | 600/38 |
| Foreign Patent | | | |
| Documents | | | |
| 2206436 | January 1989 | GB | 434/273 |

### BACKGROUND OF INVENTION

1. Field of the Invention

The field of the invention is directed to dolls and mannequins, and more specifically those made in the form of an adult human female.

2. Description of the Prior Art

Dolls have been made for thousands of years from everything from stone and wood to corn husks and today from vinyl and other polymers. Although today's fashion dolls are real looking, they are not extremely realistic.

In recent years, numerous “inflatable” dolls have been produced for use by human males for sexual release, but they are not anatomically correct and are featureless and unrealistic.

Mannequins as well have been made for as long as merchants have sold pre-made clothing. Early mannequins were carved from wood or formed with wire frames. Today's models of vinyl or other polymers are much improved, but still are not that life-like and seldom able to be posed.

The motion picture industry has used ‘body doubles’ constructed of silicone polymers for special effects for many years but these are as heavy as or heavier than an actual human body and the cost of material is exorbitant. A company known as Abyss Creations has produced these for sale to the public under the name “RealDoll” as a sex doll. The price is approximately $6000, out of reach for most individuals. The product has been in production in 1996, about the same time I documented my first drawings using certified mail. This product is not patented.

The closest patent to my appliance is U.S. Pat. No. 5,466,235, filed in 1995, which has several technical flaws and is just a human female torso. The primary flaw is the saturation of the polyurethane material with mineral oil, which would dissolve the polymer and would also contaminate fluid samples for medical use or analysis.

There is a viable need for the adult human female facsimile. The first function would be seen as a sexual appliance for the human male, but with adaptations, other uses are numerous. In addition to simple sexual release in today's world of AIDS and a number of other STD's that are incurable, the idea of a sexual outlet without the chance of contracting or transmitting a disease is desirable. Also for those that are paralyzed or have other disabilities that prevent the pursuit of or attraction of a mate or without access to a female or if used to deter rape or molestation by psychologically afflicted individuals are all possible uses. Other possible applications of this device include, but are not limited to: collection of sperm for sperm banks or for medical testing by the medical community; as a mannequin for clothing displays in department stores; for classroom study in science, biology or health classes; for CPR and first aid training; or as a model for art classes; or even as an auto or house companion to deter criminals such as burglars and car-jackers. A human male facsimile is also a viable device.

### BRIEF SUMMARY OF INVENTION

The human female facsimile appliance is an anatomically correct adult human female, life sized doll or mannequin, consisting of a rigid skeletal system, less complex than, but similar to the actual human skeleton; made of PVC, nylon or other similar polymer (or metal if strength and/or weight is needed, or for robotic or automatonic additions). Skeletal joints are steel compression joints with nylon bushings to hold a pose. The skeletal structure is covered by a tissue layer of polyurethane or similar polymer to equate the properties of human tissue, including density and heat retention, but is considerably lighter. The outer skin covering is of silicone, Dow Corning Silastic Q7-2213 or other elastimeric polymer to mimic the properties of human skin; including a flexible, soft, smooth texture that warms to the touch.

The shape of the appliance is best achieved by using a human female to cast molds for the tissue layer. The appliance is equipped with three anatomically correct orifices (vagina, anus and mouth) to accept an external cylindrical object such as the human male penis.

Other additions can be included in the appliance to increase it's desirability; such as articulated fingers, vibrators for the orifices, suction unit for the mouth, simulated breathing and heartbeat, an articulated mechanical device to move the lower abdomen, to enhance sexual pleasure. A voice unit that could be a basic recorded voice or a complex interactive computer program connected to a PC via cable or an infrared or radio transmitter. This device could be so complex that sensors could be placed at the orifices to sense sexual activity and activate an increase of breathing, heartbeat, activation of abdominal motion device and ‘passion’ induced talk would ensue. Additionally, a rudimental interactive program could allow simple to complex conversation before or after sexual activity to create a more complete ‘companion’. In reference to U.S. Pat. No. 4,683,669; facial expressions could also be added to this program.

Although the primary objective of the appliance is for sexual outlet for the human male, there are a multitude of other uses for this device. This may be a critical step toward automatonic robotics.

### BRIEF DESCRIPTION OF THE SEVERAL VIEWS OF THE DRAWINGS

FIG. 1 is a front view of the completed appliance.

FIG. 2 is a left side view of the completed appliance.

FIG. 3 is a rear view of the completed appliance.

FIG. 4 is a left side cross section view of the appliance with details of the three orifices.

FIG. 5 is a left side view of the appliance showing the mold divisions.

FIG. 6 is a view of the back of the left hand showing the skeletal and joint system of the articulated hand and wrist of the appliance.

FIG. 7 is a view of the left side of the left foot showing the skeletal and joint system of the articulated foot and ankle of the appliance.

(*FIGS. 8, 9, 10, 11, and 12 represented as 3/4 inch in diameter.)

FIG. 8 is a schematic of the details of the articulated joints, showing the components used in the construction of FIGS. 9, 10, and 11; used in the appliance.

FIG. 9 is a schematic of the 90 degree articulated joint to be used for the knees and elbows of the appliance.

FIG. 10 is a schematic of the 180 degree articulated joint to be used for the ankles, and the mid body (2) of the appliance.

FIG. 11 is a schematic of the double articulated joint to be used for the hips, shoulders, neck and wrists of the appliance.

FIG. 12 is a schematic of the rotating articulated joint to be used in the mid forearms and neck of the appliance.

FIG. 13 is a frontal schematic view of the polymer skeletal system of the appliance.

FIG. 14 is a left side schematic view of the polymer skeletal system of the appliance.

FIG. 14A is the left side schematic view of the polymer skeletal left arm as it fits on FIG. 14.

FIG. 15 is a front view of a proposed possible configuration of the completed appliance.

FIG. 16 is a left side view of a proposed possible configuration of the completed appliance.

### DETAILED DESCRIPTION OF THE INVENTION

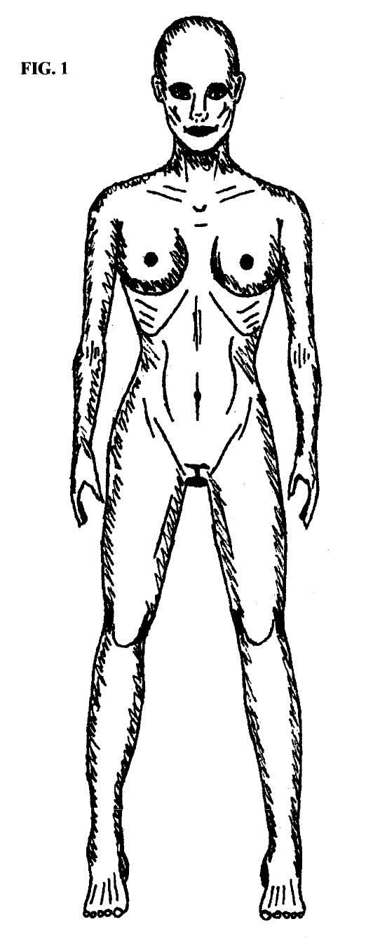

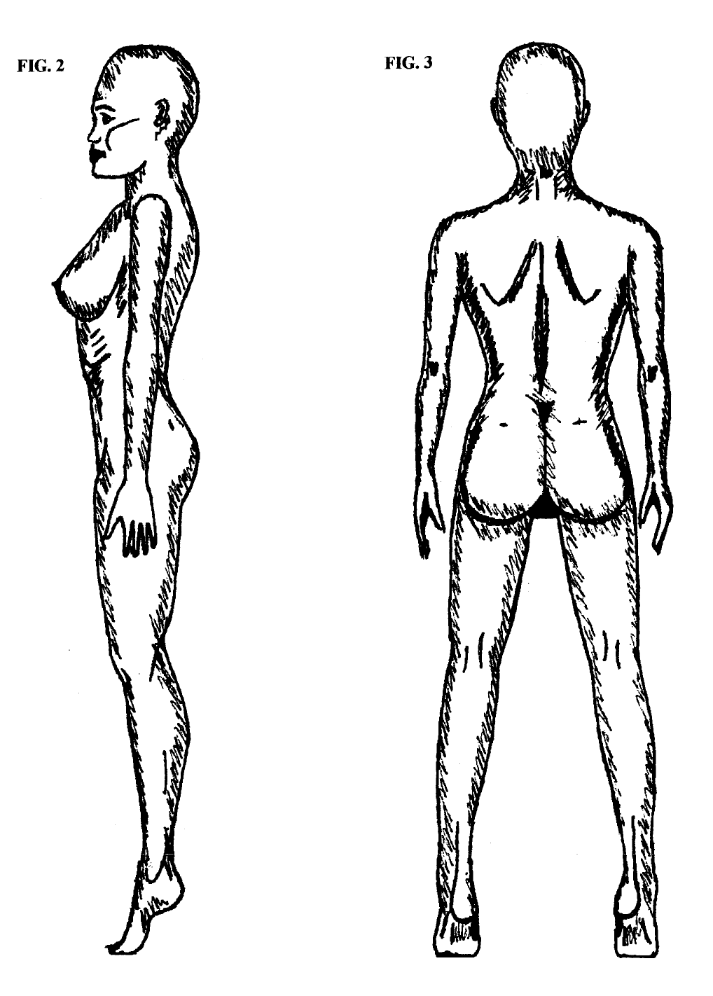

In reference to the drawings, FIGS. 1, 2, and 3 are views of a completed human female facsimile; front, left side and rear, it is in the form of a 5 foot 1 inch tall adult human female (on it's toes it is 5′ 5″ tall). This appliance can be made any dimension or shape, from a thin, petite form to a tall, heavy form or anything in between. The appliance could also be scaled to a child's doll, as well. The coating that is made of Dow Corning Silastic Q7-22 13 silicone or a similar elastimeric polymer. The coating is applied at a thickness of approximately 1/8th of an inch in thickness. It is flexible, smooth and warms to the touch as human skin. The appliance has an estimated approximate weight of 40 to 60 pounds, depending on the material used for the skeletal system, the material and density of the polymer used for the tissue layer, the thickness and material used for the skin covering and the addition of enhancement accessories used.

The skin covering can be tinted to fair, tanned, olive or ebony; tan lines can be included. The face can be made to look like any female, the features can be altered to any taste. Permanent ‘make-up’ can be applied to the face, freckles, birth marks or tatoos can be added to suit the tastes of the individual.

Breasts can be of any size and may be fluid filled with silicone, jelled saline, glycerine or keratin peptide envelopes to mimic the natural look and feel of the human female breasts. The most anatomically accurate shape will result from casting the form from a real model.

The eyes are glass or acrylic spheres inserted into eye socket openings and can be any color; blue, green, grey or brown to suit the individual. Fingernails and toenails are acrylic and can be of any style, length or color desired. Eye lashes and eye brows are imbedded into the elastimer and are permanent. Pubic hair will be applied as the eye lashes and eye brows and can be any color or style; natural, groomed or shaved. Hair can be any style or color and can either be applied permanently or as a wig, that can be changed at will.

Ear rings and body piercings can be added to accommodate the taste of the individual. Rings, necklaces or other jewelry can be used on the appliance. The appliance can be dressed it any type of clothing, from lingerie, to jeans or dress clothes and can wear shoes or boots of any type.

The appliance can be posed to any normal human position and if weighted correctly will stand unassisted.

In reference to the drawing, FIG. 4 is a left side cross section view of the appliance with details of the three orifices. The mouth includes teeth and tongue of soft material and has an opening in the back of the head and skull to allow the orifice to be cleaned with mild soap, water from a hose attached to a sink and a ‘jar mop’ type cleaning tool.

The vagina and anal orifices are actually connected, forming a single channel that allows for cleaning as with the mouth orifice. It is important that the appliance is cleaned properly after each use to prevent bacterial growth to avoid disease and unpleasant odors. A water based lubricant can be used to make use more comfortable and real feeling and will wash clean with no residue.

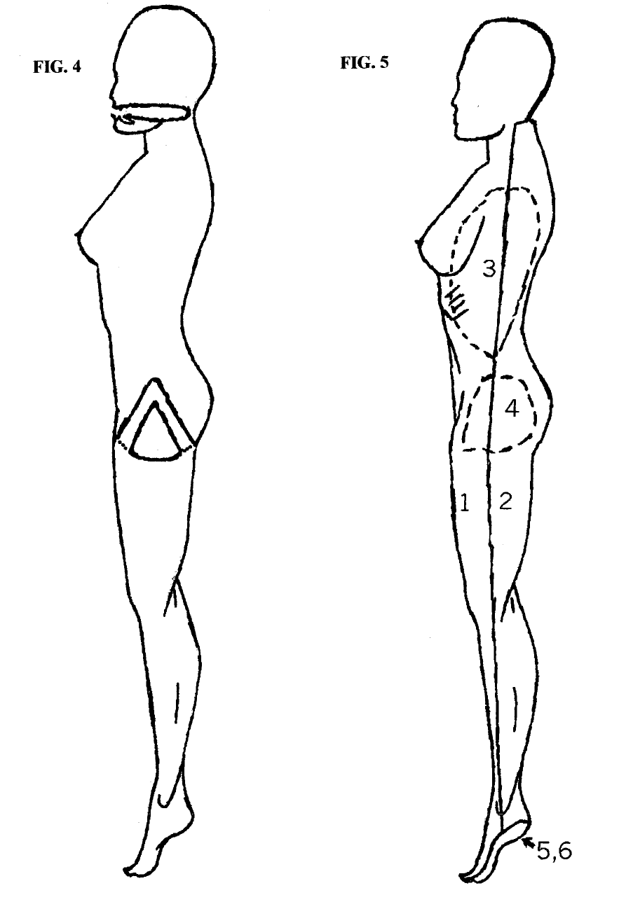

In reference to the drawing, FIG. 5 is a left side view of the appliance showing the mold divisions. The polyurethane type material for the tissue layer will be molded in 6 pieces; front, back, upper inner abdomen, lower inner abdomen and the two feet bottoms. Not shown are the following details: The eye sockets and orifices will use inserts for molding the parts. The channels for the skeletal system must be molded to fit. If breast implant envelopes are used, inserts must be used during molding to create openings for the inserts. If the addition of voice unit, computer receiver, vibrators, simulated breathing or heartbeat, mechanical abdominal device or vacuum for a suction mouth and a battery pack are used, the areas for their placement must be included in the molding process.

In reference to the drawing, FIG. 6 is the back side of the left hand of the appliance, showing the skeletal system and joints. The body of the hand is a 1/4 inch acrylic plate; attached to a double articulated joint for the wrist, which has been altered to attach to the hand. The fingers are ⅛th inch copper wire mounted in drilled holes in the plate with epoxy and hard plastic sleeves are epoxied into place, leaving exposed wire at the joints to allow the fingers to be posed like a human hand.

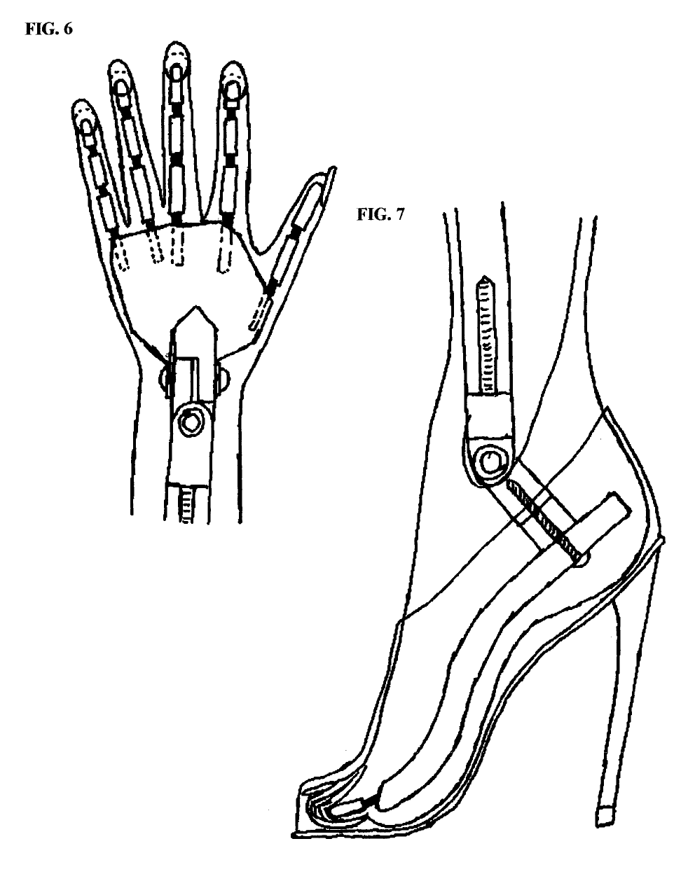

In reference to the drawing, FIG. 7 is a view of the left side of the left foot of the appliance in a shoe, showing the skeletal system and joints. The body of the foot is a ⅜th inch acrylic curved plate; attached to a 180 degree articulated joint for the ankle, which has been altered to attach to the foot. The toes are 3/32nd inch copper wire mounted in drilled holes in the plate with epoxy and hard plastic sleeves are epoxied into place, leaving exposed wire at the joints to allow the toes to be posed like the human foot.

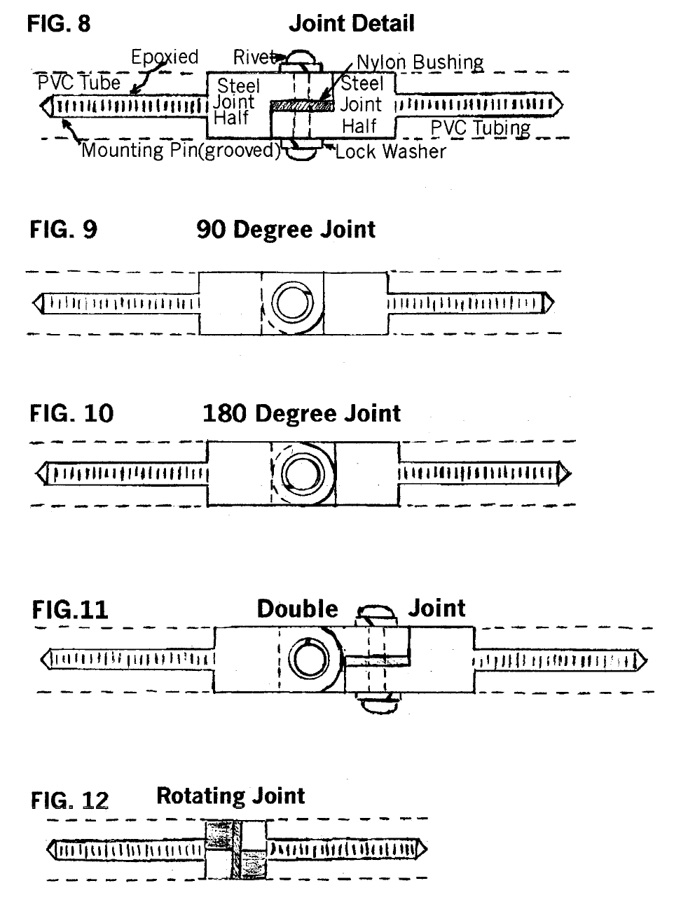

In reference to the drawing, FIG. 8 is a schematic of details of the construction of FIGS. 9, 10 and 11. This configuration will allow the joints to move and hold a pose in any desirable position. All of these joints are constructed of steel with a nylon washer to allow smooth motion, two lock washers on the outside to maintain pressure on the joint to keep it in position and all is riveted together for durability. All of these joints are based on a 3/4 inch configuration, they could be made larger or smaller to accommodate weight or other considerations. They can also be adjusted, at the time of manufacture, to move harder or easier, as is needed, by altering the compression weight on the rivet.

In reference to the drawing, FIG. 9 is a schematic of the 90 degree articulated joint to be used in the knees and elbows of the appliance, to mimic the motion of that of the human. This joint uses a lobe on the steel joint halves to limit the range of motion.

In reference to the drawing, FIG. 10 is a schematic of the 180 degree articulated joint to be used in the ankles and mid body of the appliance, to mimic the motion of that of a human. This joint uses rounded steel joint halves to allow a full 180 degree range of motion.

In reference to the drawing, FIG. 11 is a schematic of the double articulated joint to be used in the hips, shoulders, wrists and neck of the appliance, to mimic the movement of that of a human.

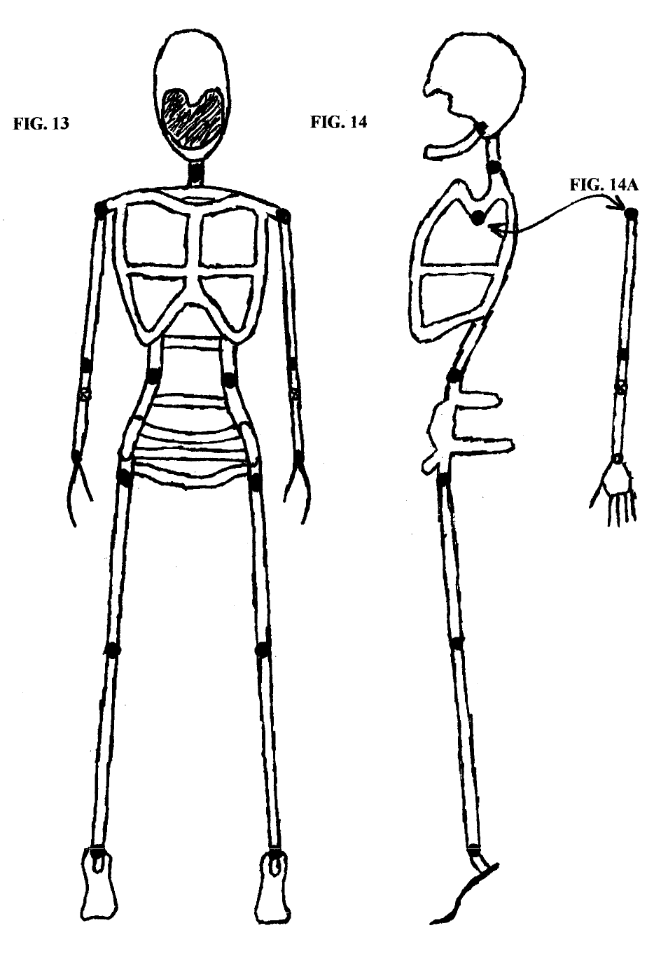

In reference to the drawing, FIG. 12 is a schematic of the rotating articulated joint to be used in the mid forearms and neck. The joint in the neck is used in conjunction with FIG. 11, the double articulated joint and has been altered to fit together. This allows the appliance to mimic the rotating motion of the human forearm and the neck. It will rotate 90 degrees as the human forearm and neck.

In reference to the drawing, FIGS. 13, 14 and 14A are frontal and left side views of the skeletal system of the appliance. The skull of the appliance is constructed of approximately 1/8th inch PVC or other polymer. The jaw is of the same material and has riveted joints similar to the compression joints (as in FIG. 8) to open and close the mouth. The skeletal system is made of PVC or other polymer and is similar to the human skeleton, but less complicated. The long ‘bones’ of the system are made of 3/4 inch tubular material (solid plastic or metal can be used for more strength or weight). The placement of the joints are marked with:●, with the exception of the rotating joints, which is marked with:

[symbol]

The skeletal system is designed to be close the surface like the human body in the following areas: skull, jaw, shoulder blades, collar bones, sternum, spine, rib cage, pelvic bones, elbows, knees, shins and knuckles.

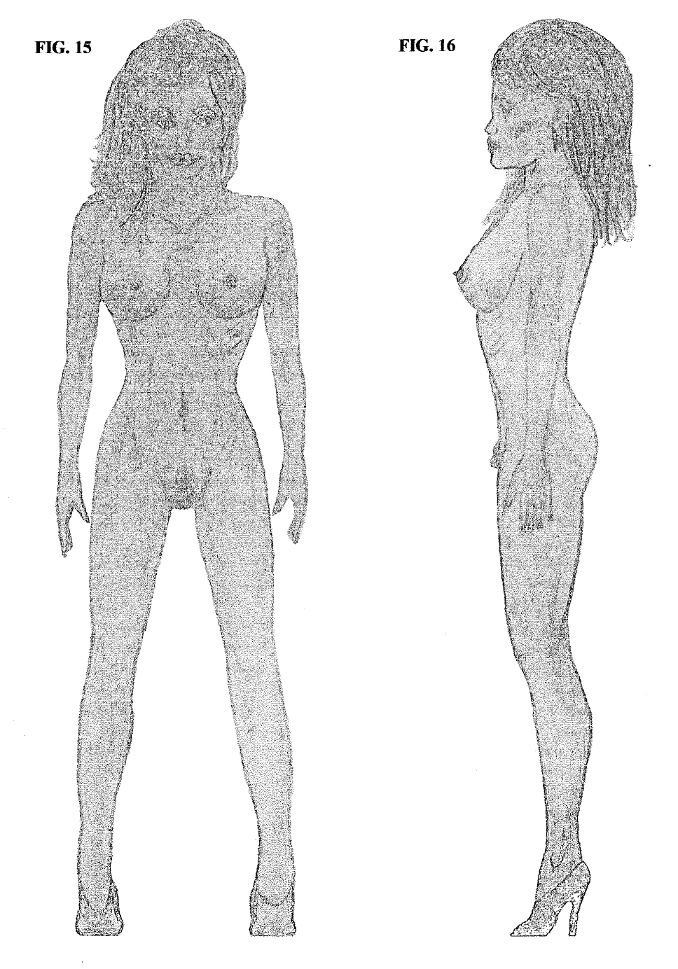

In reference to drawings FIGS. 15 and 16 are front and left side views of a proposed possible configuration of the finished appliance. With the exception of motion, the appliance will look like a living adult human female in every way. This is a single example of the endless range of possible configurations.

### Manufacturing Procedure

The construction of the human female facsimile is a labor intensive process and will require skill as well as artistic ability. The procedure will be as follows:

1. The form of the human female facsimile will be created by casting molds from one or more human females. Positive casts must be sculpted to perfection.

2. The cast will be divided into six sections for molding; the front, back, upper inner abdomen, lower inner abdomen and the bottoms of the feet. The casts will be sculpted to form eye sockets; slots for finger and toe nails; the orifices and all the additions that are to be included in the appliance. Removable inserts for these orifices will be used in the molding process. Channels for the skeletal system must be formed into the casts. Permanent molds will then be made form the casts.

3. The molds will be coated with a release agent, appropriate for the type of polymer used and the parts cast.

4. The skeletal system will be molded from PVC of other polymer to match the forms, joints will be manufactured and will be assembled.

5. The eye spheres will be formed from glass or acrylic, colored properly for the specific appliance.

6. Finger and toe nails will be molded from material tinted the correct color and style for the appliance.

7. The tissue parts will be assembled with the skeletal system and all additional features in place, using contact-type cement for the polymer of the tissue. Care must be taken not to glue the skeletal parts to the tissue so the parts will move freely inside the channels. (Tests must be done prior to manufacture to determine if slots or cut-outs will be needed at the joints, for them to bend naturally.)

8. The elastimeric polymer will be tinted to the desired color and sprayed over the tissue layer. The orifices must be coated by brushing the elastimeric polymer inside to coat the parts completely. Trimming and touch-ups will be done at this time.

9. Eye spheres, finger and toe nails will be installed and properly secured in place at this time.

10. The eye lashes and brows and pubic hair will be attached, strand at a time.

11. The ‘make-up’ coloration will be achieved by tinting the elastimeric polymer to the proper shades. It will be sprayed and/or brushed onto the surfaces. The coloration of the teeth, tongue and interior of the mouth and other orifices; the nipples of the breasts, and any wanted beauty marks, freckles, tan lines, etc. will be added in this manner at this time.

12. The hair of the head can be permanently applied in any color, length or style at this time as in step number 10, or a wig can be used to allow style and color to be changed at will.

13. The appliance will be set aside for a period of time to assure the coating is cured completely and will then be packaged for shipping.

What is claimed is a human female facsimile appliance that:

1. duplicates the form and look of an adult human female, with a;
2. skeletal system similar to that of the human, composed of solid or hollow PVC or other similar polymer material (or metal) with articulated joints to assimilate the movement of the human body, and is;
3. covered with a tissue layer, composed of polyurethane or other similar polymer to equate the density of the human body, which is;
4. covered with a skin layer of silicone or other similar poly elastimeric material, to simulate the properties of human skin, and is;
5. equipped with three orifices, as the human female, to accept an external cylindrical object, such as the human male penis.
